(19) United States
(12) Patent Application Publication
(10) Pub. No.: US 2007/0292505 A1
(43) Pub. Date: Dec. 20, 2007

(54) Controlled release alfuzosin hydrochloride formulation

(75) Inventors: Tianrun Yang, Coral Springs, FL (US); Timothy Weng, Cooper City, FL (US)

Correspondence Address: DAVIDSON, DAVIDSON & KAPPEL, LLC, 485 SEVENTH AVENUE, 14TH FLOOR, NEW YORK, NY, 10018, US

(73) Assignee: Abrika Pharmaceuticals, Inc., Sunrise, FL (US)

(21) Appl. No.: 11/453,651

(22) Filed: Jun. 15, 2006

(51) Int. Cl. A61K 9/22; A61K 31/496

(52) U.S. Cl. 424/468; 514/252.17

(57) ABSTRACT

In certain embodiments the invention is directed to a single layer controlled release pharmaceutical formulation comprising a tablet consisting of a single layer matrix comprising a therapeutically effective amount of alfuzosin or a pharmaceutically acceptable salt thereof dispersed in a controlled release material, wherein the formulation following single dose administration under fed conditions exhibits a mean time to maximum plasma concentration of about 3 hours to about 14 hours.

The present invention relates to dosage forms comprising alfuzosin or pharmaceutically acceptable salts thereof.

### BACKGROUND OF THE INVENTION

Alfuzosin is a widely used α-antagonist and is marketed as Uroxatral® 10 mg tablets by Sanofi-Synthelabo.

Alfuzosin is used to treat symptoms of benign prostatic hyperplasia (BPH, enlarged prostate) such as frequent, urgent need to urinate during the day and at night, weak urine stream, and difficulty urinating. Alfuzosin is in a class of medications called alpha-1 blockers. It works by relaxing the muscles in the prostate and bladder neck to allow urine to flow more easily.

The FDA publication entitled “Approved Drug Products with Therapeutic Equivalence”, commonly referred to as the “Orange Book” lists U.S. Pat. Nos. 4,661,491 and 6,149,940 as purportedly encompassing the active ingredient of Uroxatral® tablets (i.e., alfuzosin HCL).

Alfuzosin has a relatively short half-life and a more intense absorption at the duodenum-jejunum level, but the size of which decreases along the intestinal tract. Therefore, alftizosin must be administered several times a day. For this reason, the development of a pharmaceutical preparation with controlled release in the proximal upper parts of the tract (duodenum and jejunum) is desired.

Controlled release alfuzosin formulations have been described in U.S. Pat. Nos. 6,149,940 and 5,589,190 and European Patent No. 700 285 B1.

There continues to exist a need in the art for a controlled release dosage form of alfuzosin hydrochloride.

### OBJECTS AND SUMMARY OF THE INVENTION

It is an object of the present invention to provide a single-layer controlled release oral dosage form for alfuzosin or a pharmaceutically acceptable salt thereof.

It is a further object of certain embodiments of the present invention to provide a method for preparing a controlled release oral dosage form containing alfuzosin or a pharmaceutically acceptable salt thereof, as disclosed herein.

It is a further object of certain embodiments of the present invention to provide a method of treatment of benign hypertrophy of the prostate via administration of alfuzosin or a pharmaceutically acceptable salt thereof in a controlled release oral dosage form to a human patient in need of such treatment, as disclosed herein.

It is a further object of certain embodiments of the present invention to provide a method of treatment of hypertension, pheochromocytoma, shock and peripheral vascular diseases via administration of alfuzosin or a pharmaceutically acceptable salt thereof in a sustained release oral dosage form to a human patient in need of such treatment, as disclosed herein.

In accordance with the above objects, the present invention is directed, in part, to a single layer controlled release pharmaceutical formulation comprising: a tablet consisting of a single layer matrix comprising a therapeutically effective amount of alfuzosin or a pharmaceutically acceptable salt thereof dispersed in a controlled release material; wherein the tablet following single dose administration under fed conditions exhibits a mean time to maximum plasma concentration of about 3 hours to about 14 hours, about 4 hours to about 12 hours, or about 6 hours to about 10 hours.

In certain embodiments, the present invention is directed to a single layer controlled release pharmaceutical formulation comprising: a single layer tablet comprising a therapeutically effective amount of alfuzosin or a pharmaceutically acceptable salt thereof dispersed in a controlled release material; wherein the tablet following single dose administration under fed conditions exhibits a mean time to maximum plasma concentration of about 3 hours to about 14 hours, about 4 hours to about 12 hours, or about 6 hours to about 10 hours.

In other embodiments, the present invention is directed to a single layer controlled release pharmaceutical formulation consisting essentially of a single layer tablet comprising a therapeutically effective amount of alfuzosin or a pharmaceutically acceptable salt thereof dispersed in a controlled release material; wherein the tablet following single dose administration under fed conditions exhibits a mean time to maximum plasma concentration of about 3 hours to about 14 hours, about 4 hours to about 12 hours, or about 6 hours to about 10 hours.

In further aspects of the present invention, the controlled release formulation is directed to a single layer controlled release pharmaceutical formulation consisting essentially of: (a) a single layer matrix comprising a therapeutically effective amount of alfuzosin or a pharmaceutically acceptable salt thereof dispersed in a controlled release material; and (b) an optional coating surrounding the matrix; wherein the formulation following single dose administration under fed conditions exhibits a mean time to maximum plasma concentration of about 3 hours to about 14 hours, about 4 hours to about 12 hours, or about 6 hours to about 10 hours.

The controlled release pharmaceutical formulation of the present invention, following single dose administration under fed conditions may exhibit a mean maximum plasma concentration of about 7 μg/mL to about 27 μg/mL, about 8 μg/mL to about 20 μg/mL, or about 10 μg/mL to about 18 μg/mL.

The controlled release pharmaceutical formulation of the present invention, following single dose administration under fed conditions may exhibit a mean AUC₀₋₂₄ of about 130 μg.hr/mL to about 405 μg.hr/mL, about 150 μg.hr/mL to about 380 μg.hr/mL or about 200 μg.hr/mL to about 320 μg.hr/mL.

In certain embodiments, the controlled release formulation of the present invention is directed to a single layer controlled release pharmaceutical formulation comprising: a tablet consisting of a single layer matrix comprising a therapeutically effective amount of alfuzosin or a pharmaceutically acceptable salt thereof dispersed in a controlled release material; wherein the formulation following multiple dose administration under fed conditions exhibits a mean time to maximum plasma concentration of about 4 hours to about 12 hours, about 6 hours to about 11 hours, or about 6 to about 10 hours.

In other embodiments, the present invention is directed to a single layer controlled release pharmaceutical formulation comprising: a single layer tablet comprising a therapeutically effective amount of alfuzosin or a pharmaceutically acceptable salt thereof dispersed in a controlled release material; wherein the tablet following multiple dose administration under fed conditions exhibits a mean time to maximum plasma concentration of about 4 hours to about 12 hours, about 6 hours to about 11 hours, or about 6 to about 10 hours.

In further aspects, the present invention is directed to a single layer controlled release pharmaceutical formulation consisting essentially of a single layer tablet comprising a therapeutically effective amount of alfuzosin or a pharmaceutically acceptable salt thereof dispersed in a controlled release material; wherein the formulation following multiple dose administration under fed conditions exhibits a mean time to maximum plasma concentration of about 4 hours to about 12 hours, about 6 hours to about 11 hours, or about 6 to about 10 hours.

In other aspects, the present invention is directed to a single layer controlled release pharmaceutical formulation consisting essentially of: (a) a single layer matrix comprising a therapeutically effective amount of alfuzosin or a pharmaceutically acceptable salt thereof dispersed in a controlled release material; and (b) an optional coating surrounding the matrix; wherein the formulation following multiple dose administration under fed conditions exhibits a mean time to maximum plasma concentration of about 4 hours to about 12 hours, about 6 hours to about 11 hours, or about 6 to about 10 hours.

The controlled release pharmaceutical formulation of the present invention, following multiple dose administration under fed conditions, may exhibit a mean maximum plasma concentration of about 8 μg/mL to about 20 μg/mL, about 10 μg/mL to about 18 μg/mL, or about 12 μg/mL to about 16 μg/mL.

The controlled release pharmaceutical formulation of the present invention, following multiple dose administration under fed conditions, may exhibit a mean AUC₀₋₂₄ of about 120 μg.hr/mL to about 275 μg.hr/mL, about 150 μg.hr/mL to about 250 μg.hr/mL, or about 175 μg.hr/mL to about 225 μg.hr/mL.

In certain embodiments, the invention is directed to a controlled release formulation comprising alftizosin or a pharmaceutically acceptable salt thereof and a controlled release material comprising a cellulose derivative of a first viscosity and a cellulose derivative of a second viscosity, wherein the tablet exhibits any of the pharmacokinetic or in-vitro characteristics disclosed herein. In certain embodiments, one or both of the cellulose derivatives is hydroxypropylmethylcellulose.

In other embodiments disclosed herein, the controlled release formulation provides an in-vitro dissolution rate using USP Apparatus 2 (paddles) at 100 rpm with Japanese sinkers in 500 mL of 0.01 N HCL of about 5% to about 25% alfuzosin or pharmaceutically acceptable salt thereof released after 1 hour; about 20% to about 40% alfuzosin or pharmaceutically acceptable salt thereof released after 3 hours; about 40% to about 60% alfuzosin or pharmaceutically acceptable salt thereof released after 6 hours; and not less than about 60% alfuzosin or pharmaceutically acceptable salt thereof released after 12 hours.

In further aspects disclosed herein, the controlled release formulation provides an in-vitro dissolution rate using USP Apparatus 2 (paddles) at 100 rpm with Japanese sinkers in 500 mL of 0.01 N HCL of about 10% to about 20% alfuzosin or pharmaceutically acceptable salt thereof released after 1 hour; about 25% to about 35% alfuzosin or pharmaceutically acceptable salt thereof released after 3 hours; about 45% to about 55% alfuzosin or pharmaceutically acceptable salt thereof released after 6 hours; and not less than about 65% alfuzosin or pharmaceutically acceptable salt thereof released after 12 hours.

The present invention is also directed, in part, to a method of preparing a controlled release pharmaceutical formulation comprising (a) forming a blend comprising a therapeutically active agent and a controlled release material (b) granulating the blend (e.g., by dry and wet granulation) (c) milling the granulated blend (d) mixing the milled blend with a pharmaceutically acceptable excipient and (e) compressing the mixture. In certain alternative embodiments, the blend of step (a) can be compressed (e.g., into slugs) prior to milling step (c). The compression step can be in place of, or in addition to, the granulation step.

The present invention is further directed, in part, to a single layer controlled release pharmaceutical formulation comprising a tablet consisting of a single layer matrix comprising about 10 mg alfuzosin hydrochloride dispersed in a controlled release material the formulation being bioequivalent to alfuzosin hydrochloride extended release tablets as approved by the FDA under NDA application no. 021287.

The term “bioequivalent” is meant for purposes of the present invention to mean that the dosage form provides an AUC (bioavailability) and Cmax (rate of absorption) of about 80% to about 125% of a reference standard, e.g., Uroxatral®.

The term “single dose administration” and “multiple dose administration” is understood to mean administration to human subjects (i.e., healthy human subjects).

### BRIEF DESCRIPTION OF THE DRAWINGS

FIG. 1 shows the dissolution profile for a formulation of the present invention, tested using USP Apparatus 2 (paddles) at 100 rpm with Japanese sinkers in 500 mL of 0.01 N HCL.

FIG. 2 shows the dissolution profile for a formulation of the present invention, tested using USP Apparatus 2 (paddles) at 100 rpm with Japanese sinkers in 500 mL of 0.01 N HCL.

### DETAILED DESCRIPTION

The present invention is directed to a single layer controlled release pharmaceutical formulation which comprises a single layer matrix comprising a therapeutically effective amount of alfuzosin or a pharmaceutically acceptable salt thereof. The formulation preferably provides for once-a-day therapy and provides a mean time to maximum plasma concentration after single dose administration of about 3 hours to about 14 hours, about 4 hours to about 12 hours, or about 6 hours to about 10 hours.

Preferably, the dosage form is bioequivalent alfuzosin hydrochloride extended release tablets as approved by the FDA under NDA application no. 021287, under the tradename Uroxatral®.

The dosage forms of the present invention preferably provide for therapeutically effective treatment of benign prostatic hyperplasia (BPH, enlarged prostate) in a human patient over a once-a-day (e.g., 24 hour) time period by providing for the controlled release of alfuzosin or a pharmaceutically acceptable salt thereof. Alfuzosin is in a class of medications called alpha-1 blockers. It works by relaxing the muscles in the prostate and bladder neck to allow urine to flow more easily.

The amount of alfuzosin hydrochloride carried in the tablet is preferably between 2.5 and 50 mg.

“Pharmaceutically acceptable salts” of alfuzosin, as used herein, is meant to encompass all pharmaceutically acceptable salts, including, but not limited to, metal salts such as sodium salt, potassium salt, cesium salt and the like; alkaline earth metals such as calcium salt, magnesium salt and the like; organic amine salts such as triethylamine salt, pyridine salt, picoline salt, ethanolamine salt, triethanolamine salt, dicyclohexylamine salt, N,N′-dibenzylethylenediamine salt and the like; inorganic acid salts such as hydrochloride, hydrobromide, sulfate, phosphate and the like; organic acid salts such as formate, acetate, trifluoroacetate, maleate, fumarate, tartrate and the like; sulfonates such as methanesulfonate, benzenesulfonate, p-toluenesulfonate, and the like; amino acid salts such as arginate, asparginate, glutamate and the like. The preferred salt form for use in accordance with the present invention is the hydrochloride salt.

A non-limiting list of suitable controlled-release materials which may be included in the matrix core (e.g., a tablet core), according to the invention includes hydrophilic and/or hydrophobic materials such as polymers, protein derived materials, waxes, shellac, gums, hydrogels, and oils such as hydrogenated castor oil and hydrogenated vegetable oil. Suitable polymers include alkylcelluloses (such as ethylcellulose), acrylic and methacrylic acid polymers and copolymers (such as Eudragit® commercially available by Rohm Pharma), alkylvinyl polymers, cellulose ethers, (such as hydroxyalkylcelluloses e.g., hydroxypropylmethylcellulose) and carboxyalkylcelluloses. Examples of acrylic and methacrylic acid polymers and copolymers include methyl methacrylate, methyl methacrylate copolymers, ethoxyethyl methacrylates, ethyl acrylate, trimethyl ammonioethyl methacrylate, cyanoethyl methacrylate, aminoalkyl methacrylate copolymer, poly(acrylic acid), poly(methacrylic acid), methacrylic acid alkylamine copolymer, poly(methyl methacrylate), poly(methacrylic acid)(anhydride), polymethacrylate, polyacrylamide, poly(methacrylic acid anhydride), and glycidyl methacrylate copolymers. Waxes include, for example, natural and synthetic waxes, fatty acids, fatty alcohols, and mixtures of the same (e.g., beeswax, carnauba wax, stearic acid and stearyl alcohol). Certain embodiments of the present invention utilize mixtures of any of the foregoing controlled release materials in the matrix core. However, any pharmaceutically acceptable hydrophobic or hydrophilic controlled-release material which is capable of imparting controlled-release of the active agent may be used in accordance with the present invention.

Cellulosic polymers which may be used in the core of the present invention include hydroxyethylcellulose hydroxypropylmethyl cellulose, hydroxypropylcellulose, sodiumcarboxymethylcellulose, and mixtures thereof. A preferred extended release carrier is hydroxypropylmethylcellulose (“HPMC”).

In preferred embodiments, the controlled release material further comprises effective amounts of different grades (e.g., different viscosities) of hydroxypropylmethylcellulose (HPMC). In certain embodiments, the HPMC utilized can be Methocel K4M® and Methocel E5® by Colorcon (West Point, Pa.).

In certain preferred embodiments, the hydrogenated vegetable oil is Lubritab®, commercially available by JRS Pharma.

In certain preferred embodiments, the ethylcellulose is Ethocel Standard®, by Dow Chemical Co., Ltd.

In certain embodiments the total content of hydroxypropylmethylcellulose is from about 50 to about 80% of the final formulation.

In certain preferred embodiments the total content of hydroxypropyl methylcellulose is from about 70% to about 80% of the final formulation.

In certain preferred embodiments the total content of the hydrogenated vegetable Oil is from about 10 to about 20% of the final formulation, preferably from about 12 to about 15%.

In certain preferred embodiments, the ethylcellulose is present in an amount of from about 1 to about 10% of the final formulation, preferably from about 3 to about 7%.

In addition to the above ingredients, in certain embodiments the controlled release matrix core of the present invention may further include a wide variety of additives and excipients that enhance drug solubility or, that promote stability, tableting or processing of the dispersion. Such additives and excipients include tableting aids, lubricants, surfactants, fillers or diluents, water-soluble polymers, pH modifiers, binders, pigments, disintegrants, glidants, plasticizer, solvents, flow conditioning agents, suspending agents, viscosity-increasing agents, anti-caking agents, antioxidants, lubricants and flavorants. Exemplary of such components are metallic salts of acids such as aluminum stearate, calcium stearate, magnesium stearate, sodium stearate, and zinc stearate; fumed or colloidal silica which is commercially available as Cab-O-Sil M5®, by Cabot Corporation; povidone, fatty acids, hydrocarbons and fatty alcohols such as stearic acid, palmitic acid, liquid paraffin, stearyl alcohol, and palmitol; fatty acid esters such as glyceryl (mono- and di-) stearates, triglycerides, glyceryl (palmiticstearic) ester, sorbitan monostearate, saccharose monostearate, saccharose monopalmitate, and sodium stearyl fumarate; alkyl sulfates such as sodium lauryl sulfate and magnesium lauryl sulfate; polymers such as polyethylene glycols, polyoxethylene glycols, and polytetrafluoroethylene; and inorganic materials such as talc and dicalcium phosphate; sugars such as lactose, xylitol, sucrose, dextrose, fructose, sorbitol, mannitol, starches, other polyols, mixtures thereof and the like; and sodium starch glycolate. The quantities of these additional materials will be sufficient to provide the desired effect to the desired formulation. Specific examples of pharmaceutically acceptable carriers and excipients that may be used to formulate oral dosage forms are described in the Handbook of Pharmaceutical Excipients, American Pharmaceutical Association (1986), incorporated by reference herein.

Examples of lubricants include stearic acid, magnesium stearate, carnauba wax, glyceryl behenate, talc, mineral oil (in PEG), mixtures thereof, and the like. Magnesium stearate and camauba wax are preferred lubricants.

Examples of binders include water-soluble polymer, such as modified starch, gelatin, polyvinylpyrrolidone, polyvinyl alcohol, povidone, sodium carboxymethylcellulose, alginic acid, poly(ethylene glycol), poly(propylene glycol), guar gum, polysaccharide, bentonite clay, sugar, poloxamer, collagen, albumin, gelatin, mixtures thereof, and the like.

Examples of fillers or diluents for use in the present invention include for example, lactose, microcrystalline cellulose, dextrin, dextrose, starch, mixtures thereof and the like.

Examples of glidants for use in the present invention include for example, calcium phosphate tribasic, calcium silicate, powdered cellulose, colloidal silicon dioxide, magnesium silicate, magnesium trisilicate, silicon dioxide, starch, talc, mixtures thereof and the like.

Direct compression vehicles may be used and include, for example, processed forms of cellulose, sugars, and dicalcium phosphate dihydrate, among others. Microcrystalline cellulose is an example of a processed cellulose that has been utilized extensively in the pharmaceutical industry as a direct compression vehicle for solid dosage forms.

In certain embodiments, the dosage form of the present invention comprises from about 1 to about 80% by weight of the dosage form of a controlled release material; from about 0.1 to about 1% by weight of the dosage form of silica gel and from about 0.5 to about 2% by weight of the dosage form of a pharmaceutically acceptable lubricant.

In certain aspects of the present invention, the dosage form has an optional coating, such as a controlled release coating, an enteric coating, or a film coat. The coating may be applied in any pharmaceutically acceptable manner known to those skilled in the art. For example, in one embodiment, the coating is applied via a fluidized bed. In another embodiment, the coating is applied via a coating pan. In certain embodiments, the coating further includes a binder as disclosed herein.

Examples of suitable enteric polymers which may be used for the optional enteric coating include cellulose acetate phthalate, hydroxypropyl-methylcellulose phthalate, polyvinylacetate phthalate, methacrylic acid copolymer, shellac, hydroxypropylmethylcellulose succinate, cellulose acetate trimellitate, and mixtures of any of the foregoing. An example of a suitable commercially available enteric material is available under the trade name Eudragit® L30D55 or Acryl-Eze®.

A film coat is designed to rapidly disintegrate or dissolve in water or the environment of use. The film coat may be a conventional sugar or polymeric film coating which is applied in a coating pan or by conventional spraying techniques. Preferred materials for the film coat are hydroxypropymethylcellulose, polyvinyl alcohol, or a mixture thereof. An example of a commercially available film coat is Opadry tradename (e.g., Opadrye® II, Yellow), from Colorcon, West Point, Pa.

The controlled release material that can be used for the optional coating may be any of the hydrophilic or hydrophobic polymers described above, such as cellulosic polymers or acrylic polymers.

### DETAILED DESCRIPTION OF THE PREFERRED EMBODIMENTS

The following example illustrates a particular aspect of the present invention. It is not to be construed to limit the claims in any manner whatsoever.

### EXAMPLE 1

The ingredients of the single layer tablet of Example 1 are set forth in Table 1 below:

TABLE 1

| | Formulation |
|---|---|
| Ingredients | Percent (%) |
| Part A | |
| Alfuzosin Hydrochloride | 2.94 |
| Hydroxypropyl- | 51.25 |
| methylcellulose | |
| (Methocel, K4M ®) | |
| Hydroxypropyl- | 25.75 |
| methylcellulose | |
| (Methocel E5 ®) | |
| Silica Gel | 0.50 |
| (Syloid ® 244FP) | |
| Magnesium Stearate | 0.25 |
| Subtotal for Part A | 80.69% |
| Part B | |
| Hydrogenated Vegetable Oil, | 14.06 |
| Type 1 | |
| (Lubritab ®) | |
| Ethylcellulose | 5.00 |
| (Ethocel ® Standard 10) | |
| Magnesium Stearate | 0.25 |
| Subtotal for Part B | 19.31% |
| TOTAL | 100 |

The controlled release formulation of Example 1 was prepared as follows:

I. Weighing and Blending:
1. Weigh out each ingredient according to the table above.
2. Into an 8 qt V-blender, add the materials from the above step and blend for 5 min.
3. Discharge contents and pass pre-blend through a Quadro Comil fitted with a 0.045″ screen.
4. Place screened blend back into the V-blender.
5. Blend for 10 minutes.
6. Screen magnesium stearate through a #20 mesh stainless steel sieve.
7. Add the magnesium stearate to the blend in the V-blender and mix for 3 min.

II. Blending/slugging for Part A:
8. Use roller compactor to produce proper slug,
9. After Slugging, use a Fitzmill L1A to mill the tablets through a 0.093″ screen.
10. Collect, weigh, and label milled blend as Part A.

III. Weighing/Blending for Part B Blend):
11. Weigh out the alfuzosin HC1 blend and each excipient according to the tables above for Part B.
12. Screen the hydrogenated vegetable oil through comil with 0.045″ screen.
13. Add the alfuzosin HC1 blend from Step 11 and the screened hydrogenated vegetable oil from the above step and ethylcellulose into an 8 qt V-blender and blend for 10 minutes.
14. Screen the magnesium stearate through a #20 mesh stainless steel sieve.
15. Add the magnesium stearate from the above step into the V-blender containing the blend from Step 13 and mix for an additional three minutes.

IV Compression
16. Compress tablets using a Korsch XL-100 set up with 9 mm diameter, B-tooling, standard concave, one station, gravity feeder.
17. On automatic mode, compress tablets with an individual tablet weight of 340 mg at the following tablet hardiness: 8 kp.

### EXAMPLE 2

In-vitro dissolution studies were performed using USP Apparatus 2 (paddles) at 100 rpm with Japanese sinkers in 500 mL of 0.01 N HCL on Alfuzosin Hydrochloride 10 mg tablets prepared in accordance with Example 1. The results are set forth in Table 2.

TABLE 2
% Dissolved Results (Cumulative Accounting for Volume Withdrawn
and Drug Removed)

| | Pull # | | | | | | |
|---|---|---|---|---|---|---|---|
| | 1 | 2 | 3 | 4 | 5 | 6 | 7 |
| | Time Point (Hr.) | | | | | | |
| Sample # | 1 | 3 | 6 | 9 | 12 | 15 | 20 |
| Vessel-1 | 15.0 | 31.6 | 50.7 | 65.5 | 77.7 | 86.1 | 96.2 |
| Vessel-2 | 15.9 | 33.0 | 52.1 | 66.9 | 78.8 | 87.3 | 97.2 |
| Vessel-3 | 14.5 | 30.2 | 48.3 | 62.2 | 73.0 | 81.4 | 91.4 |
| Vessel-4 | 15.2 | 31.5 | 49.4 | 63.1 | 74.2 | 83.0 | 92.8 |
| Vessel-5 | 15.4 | 32.0 | 50.7 | 65.0 | 76.2 | 84.8 | 94.8 |
| Vessel-6 | 15.0 | 30.2 | 46.8 | 59.4 | 69.7 | 78.1 | 88.6 |
| Mean | 15 | 31 | 50 | 64 | 75 | 83 | 94 |
| SD | 0.5 | 1.1 | 1.9 | 2.7 | 3.3 | 3.4 | 3.2 |
| Min | 15 | 30 | 47 | 59 | 70 | 78 | 89 |
| Max | 16 | 33 | 52 | 67 | 79 | 87 | 97 |
| % RSD | 3.3 | 3.5 | 3.8 | 4.2 | 4.4 | 4.1 | 3.4 |

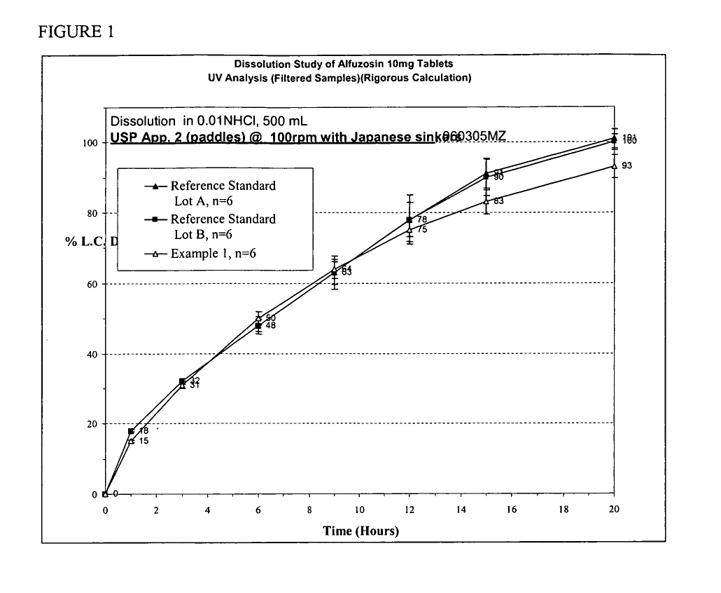

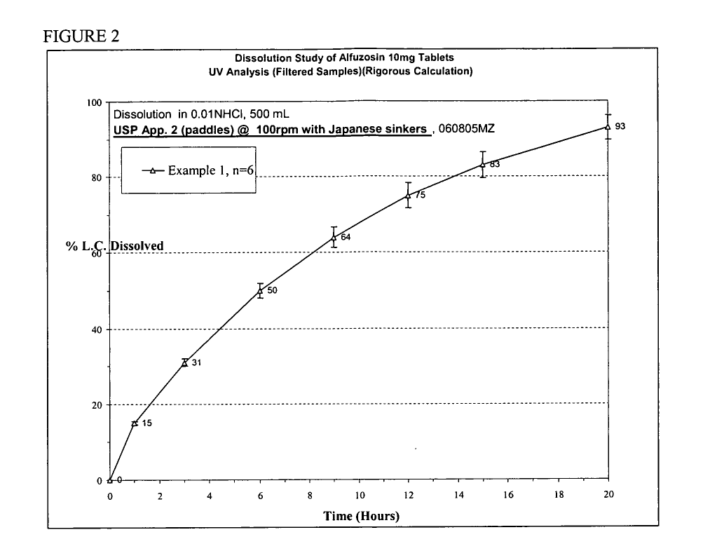

### The data of Table 2 is depicted in FIG. 1 (as compared to two lots of reference Alfuzosin Hydrochloride Extended-Release Tablets (UROXATRAL® by Sanofi-Synthelabo, Inc.)) and FIG. 2.

### EXAMPLE 3

A bioavailability study of 10 mg Alfuzosin hydrochloride extended-release tablets was performed as a randomized, single-dose, two-way crossover pilot study under fasting conditions with ten healthy male volunteers and no alternates. The two products compared were 10 mg of test Alfuzosin Hydrochloride Extended-Release Tablets (by Abrika Pharmaceuticals) or 10 mg of reference Alfuzosin Hydrochloride Extended-Release Tablets (UROXATRAL® by Sanofi-Synthelabo, Inc.) Blood samples were collected at: 1, 2, 3, 4, 5, 6, 7, 8, 9, 10, 12, 14, 16, 18, 20, 24, 48 and 72 hours.

The bioavailability study showed the following results under fasting conditions:

TABLE 3
N = 10
Ln-Transformed Data

| | Least Squares | | | | |
|---|---|---|---|---|---|
| PK | Mean | | Geometric Mean | | % |
| Variable | Test | Reference | Test | Reference | Ratio |
| Cₘₐₓ | 8.836 | 8.696 | 6874.81 | 5976.30 | 115.03 |
| AUC_{0–t} | 11.537 | 11.508 | 102479.83 | 99515.63 | 102.98 |

| | | |
|---|---|---|
| PK | Least Squares Mean | % |

| Variable | Test | Reference | Ratio |
|---|---|---|---|
| Cₘₐₓ | 7511.84 | 6584.29 | 114.09 |
| AUC_{0–t} | 114444.60 | 106796.29 | 107.16 |
| Tₘₐₓ | 4.80 | 5.20 | 92.31 |
| kₑ | 0.0312 | 0.0556 | 56.12 |
| t_{1/2} | 32.75 | 12.06 | 271.45 |

The bioavailability study showed the following results under fed conditions:

TABLE 4
N = 10
Ln-Transformed Data

| | Least Squares | | | | |
|---|---|---|---|---|---|
| PK | Mean | | Geometric Mean | | % |
| Variable | Test | Reference | Test | Reference | Ratio |
| Cₘₐₓ | 9.701 | 9.770 | 16329.20 | 17495.58 | 93.33 |
| AUC_{0–t} | 12.380 | 12.501 | 238061.52 | 268590.57 | 88.63 |

| | | |
|---|---|---|
| PK | Least Squares Mean | % |

| Variable | Test | Reference | Ratio |
|---|---|---|---|
| Cₘₐₓ | 17566.93 | 19753.30 | 88.93 |
| AUC_{0–t} | 259836.90 | 297901.52 | 87.22 |
| Tₘₐₓ | 6.70 | 9.00 | 74.44 |
| kₑ | 0.0855 | 0.0761 | 112.29 |
| t_{1/2} | 8.50 | 9.22 | 92.09 |

1. A single layer controlled release pharmaceutical formulation comprising: a tablet consisting of a single layer matrix comprising a therapeutically effective amount of alfuzosin or a pharmaceutically acceptable salt thereof dispersed in a controlled release material; wherein the tablet following single dose administration under fed conditions tio human subjects exhibits a mean time to maximum plasma concentration of about 3 hours to about 14 h ours.

2-3. (canceled)

4. A single layer controlled release pharmaceutical formulation consisting essentially of:
(a) a single layer matrix comprising a therapeutically effective amount of alfizosin or a pharmaceutically acceptable salt thereof dispersed in a controlled release material; and
(b) an optional coating surrounding the matrix;
wherein the formulation following single dose administration under fed conditions to human subjects exhibits a mean time to maximum plasma concentration of about 3 hours to about 14 hours.

5. The controlled release pharmaceutical formulation of claim 1, wherein the formulation following single dose administration under fed conditions exhibits a mean maximum plasma concentration of about 7 μg/mL to about 27 μg/mL.

6. The controlled release pharmaceutical formulation of claim 5, wherein the formulation following single dose administration under fed conditions exhibits a mean maximum plasma concentration of about 8 μg/mL to about 20 μg/mL.

7. The controlled release pharmaceutical formulation of claim 6, wherein the formulation following single dose administration under fed conditions exhibits a mean maximum plasma concentration of about 10 μg/mL to about 18 μg/mL

8. The controlled release pharmaceutical formulation of claim 1, wherein the formulation following single dose administration under fed conditions exhibits a mean AUC0-24 of about 130 μg.hr/mL to about 405 μg.hr/mL.

9. The controlled release pharmaceutical formulation of claim 8, wherein the formulation following single dose administration under fed conditions exhibits a mean AUC0-24 of about 150 μg.hr/mL to about 380 μg.hr/mL.

10. The controlled release pharmaceutical formulation of claim 9, wherein the formulation following single dose administration under fed conditions exhibits a mean AUC0-24 of about 200 μg.hr/mL to about 320 μg.hr/mL.

11-14. (canceled)

15. The controlled release pharmaceutical formulation of claim 1, wherein the formulation following multiple dose administration under fed conditions exhibits a mean maximum plasma concentration of about 8 μg/mL to about 20 μg/mL.

16. The controlled release pharmaceutical formulation of claim 5, wherein the formulation following multiple dose administration under fed conditions exhibits a mean maximum plasma concentration of about 10 μg/mL to about 18 μg/mL.

17. The controlled release pharmaceutical formulation of claim 6, wherein the formulation following multiple dose administration under fed conditions exhibits a mean maximum plasma concentration of about 12 μg/mL to about 16 μg/mL

18. The controlled release pharmaceutical formulation of claim 1, wherein the formulation following multiple dose administration under fed conditions exhibits a mean AUC0-24 of about 120 μg.hr/mL to about 275 μg.hr/mL.

19. The controlled release pharmaceutical formulation of claim 1, wherein the formulation following multiple dose administration under fed conditions exhibits a mean AUC0-24 of about 150 μg.hr/mL to about 250 μg.hr/mL.

20. The controlled release pharmaceutical formulation of claim 1, wherein the formulation following multiple dose administration under fed conditions exhibits a mean AUC0-24 of about 175 μg.hr/mL to about 225 μg.hr/mL.

21. The controlled release pharmaceutical formulation of claim 1, further comprising a coating surrounding the tablet.

22. The controlled release pharmaceutical formulation of claim 1, wherein the controlled release material is selected from the group consisting of alkylcelluloses, acrylic and methacrylic acid polymers and copolymers, cellulose ethers, hydroxyalkylcelluloses, carboxyalkylcelluloses, waxes, gums, and mixtures thereof.

23. The controlled release pharmaceutical formulation of claim 22, wherein the controlled release material comprises hydroxypropylmethylcellulose.

24. The controlled release pharmaceutical formulation of claim 1 wherein the process for preparing the tablet comprises (a) forming a blend comprising the alfuzosin or pharmaceutically acceptable salt thereof and the controlled release material; and (b) granulating the blend.

25. The controlled release pharmaceutical formulation of claim 24, wherein the process for preparing the tablet further comprises (c) milling the granulated blend of alfuzosin or pharmaceutically acceptable salt thereof and the controlled release material; (d) mixing the milled blend with a pharmaceutically acceptable excipient; and (e) compressing the mixture.

26. The controlled release pharmaceutical formulation of claim 25, wherein the pharmaceutically acceptable excipient of step (d) comprises a controlled release material.

27. The controlled release pharmaceutical formulation of claim 26, wherein the controlled release material is selected from the group consisting of alkylcelluloses, acrylic and methacrylic acid polymers and copolymers, cellulose ethers, hydroxyalkylcelluloses, carboxyalkylcelluloses, waxes, gums, and mixtures thereof.

28. The controlled release pharmaceutical formulation of claim 27, wherein the controlled release material comprises ethylcellulose, hydrogenated vegetable oil, or a mixture thereof.

29. A method of treating benign prostatic hyperplasia comprising administering to a patient in need thereof, a pharmaceutical formulation of claim 1.

30-36. (canceled)

37. A single layer controlled release pharmaceutical formulation comprising:
a tablet consisting of a single layer matrix comprising about 10 mg alfuzosin hydrochloride dispersed in a controlled release material;
wherein the formulation is bioequivalent to alfuzosin hydrochloride extended release tablets as approved by the FDA under NDA application no. 021287.

38-43. (canceled)

44. The controlled release formulation according to claim 1, which provides an in-vitro dissolution rate using USP Apparatus 2 (paddles) at 100 rpm with Japanese sinkers in 500 mL of 0.01 N HCL of about 5 to about 25% alfuzosin or pharmaceutically acceptable salt thereof released after 1 hour; about 20% to about 40% alfuzosin or pharmaceutically acceptable salt thereof released after 3 hours; about 40% to about 60% alfuzosin or pharmaceutically acceptable salt thereof released after 6 hours; and not less than about 60% alfuzosin or pharmaceutically acceptable salt thereof released after 12 hours.

45. The controlled release formulation according to claim 44 which provides an in-vitro dissolution rate using USP Apparatus 2 (paddles) at 100 rpm with Japanese sinkers in 500 mL of 0.01 N HCL of about 10 to about 20% alfuzosin or pharmaceutically acceptable salt thereof released after 1 hour; about 25% to about 35% alfuzosin or pharmaceutically acceptable salt thereof released after 3 hours; about 45% to about 55% alfuzosin or pharmaceutically acceptable salt thereof released after 6 hours; and not less than about 65% alfuzosin or pharmaceutically acceptable salt thereof released after 12 hours.

46-50. (canceled)
